(19) United States
(12) Patent Application Publication
(10) Pub. No.: US 2022/0233728 A1
(43) Pub. Date: Jul. 28, 2022

(54) In Situ Promotion of Cellular Structure By Selective Application of Electro-Magnetic Waves

(71) Applicant: BECC Applied Sciences, L.L.C., SOLON, IA (US)

(72) Inventors: Kimberly Morio, Solon, IA (US); Robert Sternowski, Cedar Rapids, IA (US)

(73) Assignee: BECC Applied Sciences, L.L.C., SOLON, IA (US)

(21) Appl. No.: 17/716,429

(22) Filed: Apr. 8, 2022

Related U.S. Application Data
(63) Continuation of application No. PCT/US2020/055061, filed on Oct. 9, 2020.
(60) Provisional application No. 62/913,831, filed on Oct. 11, 2019.

(51) Int. Cl. A61L 2/00

(52) U.S. Cl. CPC A61L 2/0047; A61L 2430/34; A61L 2202/11; A61L 2202/14

(57) ABSTRACT

A housing, a UVC emitting light source combined to the housing for emitting UVC light outside the housing onto the area of interest, and a controller combined to the UVC light source for controlling the intensity of the UVC light emitted onto the area of interest.

### CROSS-REFERENCE TO RELATED APPLICATIONS

This application is a continuation of international patent application PCT/US2020/055061, filed on Oct. 9, 2020 designating the U.S., which claims the benefit of U.S. Provisional Patent Application No. 62/913,831 filed Oct. 11, 2019, which is incorporated herein by reference.

### TECHNICAL FIELD

The present disclosure relates to disinfectants and sterilization, and more specifically, this disclosure relates to the use of specifically selected narrow-wavelength electro-magnetic waves application to cellular structures.

### BACKGROUND INFORMATION

The impact of the portion of the electro-magnetic wave spectrum encompassing light on living cells is well-known, from observations collected over thousands of years. These observations include photosynthesis, stimulation of vitamin D production, and even sunburn and blistering. In all cases, the wavelength of the light is approaching the physical size of the cellular structure. Absorption of the light power by those features can cause retardation and/or growth of chemical bonds and reactions, which is the basis for the aforementioned phenomena.

It is similarly well-known that specific wavelengths produce specific effects. For example, of the broad solar light spectrum impinging on Earth from the Sun, the short-wavelength ultra-violet (UV) sub-band is the most damaging. It has been known for some time that ultraviolet (UV) light, however, can also have antimicrobial effects. Early experiments demonstrated that properties of sunlight (either a heating effect or a property of the sun's rays itself) could prevent bacterial growth. Later, UV light was shown to be bacteriocidal to many bacteria, including Mycobacterium tuberculosis, Staphylococcus, Streptococcus, Bacillus anthrasis, and Shigella dysenteriae. UV light has also been a common treatment for tuberculosis of the skin.

UV light can be divided into different classes based on wavelength, including ultraviolet A (WA) at about 350 nm, ultraviolet B (UVB) at about 300 nm, and ultraviolet C (UVC) at about 250 nm. Not unexpectedly, the effectiveness of UV light in producing biological changes can differ at different wavelengths.

For wound healing, the use of UVC light is attractive in that it is a non-pharmacological treatment that is non-invasive to the wound. It has been demonstrated that UV light can increase epithelial cell turnover, release prostaglandin precursors and histamines, increase vascular permeability, accelerate DNA synthesis, and inactivate bacterial cells. However, UVA and UVB have been shown to cause damage to the skin, particularly in the form of sunburn and blistering, each of which would be undesirable, particularly to an open wound; also, these forms of UV radiation have been demonstrated to be carcinogenic.

Accordingly, there is a need for a device that can apply specifically selected narrow-wavelength electro-magnetic waves to the cellular structures of an area or volume of interest.

It is also well-known that specific wavelengths of radio signals—much longer in wavelength than light—produce unique effects on tissue as a function of time, frequency and power. A prominent example is microwave heating by 2450 MHz radio energy as used in common microwave ovens. 2450 MHz is the resonant frequency of water molecules, and hence applying radio energy of that frequency to any water-containing material (food, living tissue, a bottle of water, etc.) may be best visualized as causing the water molecules to vibrate and heat each other from friction. For exemplary purposes, the following discussion will focus on the application of light wavelengths but recognizing that the principles apply equally to all of the electromagnetic spectrum.

### SUMMARY

In accordance with one aspect of the present invention, there is disclosed a device for disinfecting and promoting tissue growth. The device comprises of a housing, a UVC emitting light source combined to the housing for emitting UVC light outside the housing onto the area of interest, and a controller combined to the UVC light source for controlling the intensity of the UVC light emitted onto the area of interest.

In one implementation for treating an area of interest, the housing further comprises a front-facing surface for orienting toward the area of interest, wherein the front-facing surface comprises of a depression that has positioned therein the UVC emitting light source. A pair of visible light sources can be positioned in the depression of the front-facing surface of the housing on opposite sides of the UVC emitting light source for providing visible lighted boundaries for the UVC light.

A power source for providing power to the UVC emitting light source and a switch positioned electrically between the power source and the UVC emitting light source is provided for selectively turning on and off the UVC emitting light source. The UVC emitting light source can be a UVC light-emitting diode (LED). In such instances, a constant current device electrically connected between the power source and the UVC LED for controlling the current to the UVC LED. In some implementations, a plurality of parallel-connected UVC LEDs can each controlled by one of a corresponding plurality of parallel-connected constant current devices. A pair of visible light sources on opposite sides of the plurality of UVC LEDs can be provided for visible boundaries for the UVC light and warning of the UVC light. In other implementations, a plurality of UVC emitting light sources with each having a spectral output centered around a different specific wavelength is provided.

Also, each of the plurality of UVC emitting light sources can be connected to its own controller for independent on/off control. Furthermore, in some implementations, the constant current device is a milliamp current to the constant current device, which is reducible by an analog voltage or a pulse-width modulated signal to the constant current device, and where the light source is a light emitting diode and the constant current device provides a constant current to the light emitting diode throughout the temperature range of the light emitting diode. The controller can further comprise of a duty cycle controller to modify a pulsating on/off rate of the light source.

In yet other implementations, the device can comprise a plurality of UVC emitting light sources connected in parallel; a front-facing surface with a depression extending along the front-facing surface with the plurality of UVC emitting light sources positioned in a row in the depression; a pair of visible light sources positioned on opposite sides of the UVC emitting light source for providing visible lighted boundaries for the UVC light; and a plurality of constant current devices each one of which is connected to a corresponding one of the plurality of UVC emitting light sources to provide a constant current to the corresponding UVC emitting light source throughout the temperature range of the UVC emitting light source.

In yet another implementation, the device can comprise a light conduit in electromagnetic communication with the light source for communicating light from the light source to the area of interest. The light conduit can comprise a core extending the length of the conduit; and a cladding surrounding the core up to a distal end of the core, wherein a surface of the core is exposed at the distal end, and wherein light from a light source that emits light propagates through the core and is emitted from the core along the distal end of the core. The light conduit can be provided as a plastic fiberoptic conduit comprising a cladding that has been removed from the distal end so that light is emitted radially and axially relative to the distal end of the conduit for insertion into a volume of interest for treatment. The light conduit can comprise a cylindrical distal end and wherein light radiates from the cylindrical distal end in three dimensions, and wherein the light conduit comprises of a transverse core that channels light from the light source therethrough and a cladding layer surrounding the core to the beginning of the cylindrical distal end to block light from exiting the core before reaching the cylindrical distal end, wherein the cladding layer is removed from the light conduit at the cylindrical distal end, and wherein the cylindrical distal end is a portion of the core of the light conduit without the cladding layer. The UVC emitting light source can be a UVC light-emitting diode (LED). The constant current device can be electrically connected between the power source and the UVC LED for controlling the current to the UVC LED. The constant current device can be a milliamp current to the constant current device, which is reducible by an analog voltage or a pulse-width modulated signal to the constant current device, and where the light source is a light emitting diode and the constant current device provides a constant current to the light emitting diode throughout the temperature range of the light emitting diode, and wherein the controller further comprises of a duty cycle controller to modify a pulsating on/off rate of the light source.

### BRIEF DESCRIPTION OF THE DRAWINGS

These and other features and advantages of the present invention will be better understood by reading the following detailed description, taken together with the drawings wherein:

FIG. 1 is a portable handheld device for disinfecting an area of interest.

FIG. 2 is the device of FIG. 1 from the front-face perspective.

FIG. 3 is an electrical schematic for the device of FIG. 1

FIG. 4 shows a graph of area of inhibition versus time duration for six common microbes.

FIG. 5 is a portable handheld device for disinfecting a volume of tissue.

FIG. 6 shows section A-A from FIG. 5.

FIG. 7 shows an electrical schematic for device 200.

### DETAILED DESCRIPTION OF THE PREFERRED EMBODIMENTS

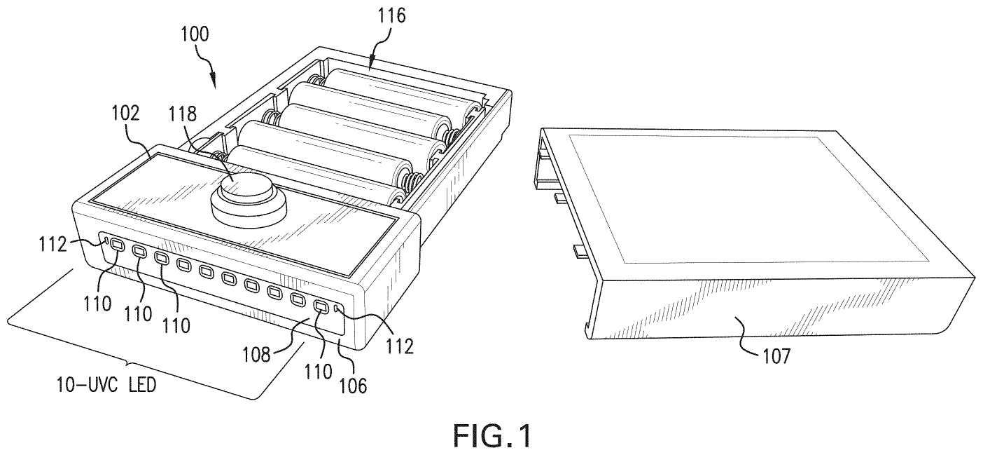

Referring to FIG. 1, shown is a device 100 for disinfecting an area of interest in accordance with the present disclosure. Device 100 emits electro-magnetic waves in the ultra-violet C spectrum to alter the structure of cells in the area of interest. Device 100 comprises of at least one light source 110 for generating the light in the spectrum of interest, which preferably has a narrow-wavelength in the 200-280 nm range, including any range of values or any specific value within that range. While light source 110 can be implemented as a solid state laser, microwave generated UV plasma, or any type of fixed or tunable wavelength source, light source 110 is hereinafter described as a light emitting diode (LED). Also, for purposes of this disclosure, a topical application of light will be described, although one skilled in the art will recognize that a non-topical application is also contemplated.

The light from device 100 is applied to the area of interest to achieve cellular stimulation in the illuminated area. The amount and rate of stimulation is a function of the wavelength, light power, and time duration of the application. The amount of light power may be varied electronically by a light source adjustment or by varying the distance from the light source focal point to the area of interest.

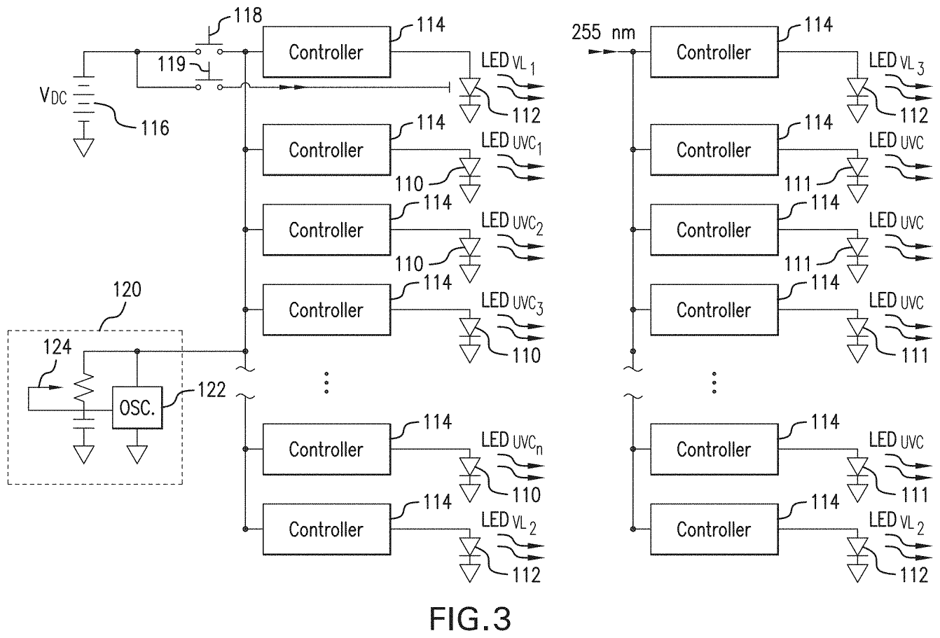

Referring to FIG. 3, shown is an electrical schematic for implementing device 100. Device 100 has n-number of light sources 110 shown as LED_{UVC1}-LED_{UVCn}. Each light source 110 is a LED that emits UVC light in the spectrum of interest. In one implementation, each n-number of light sources 110 can have the same wavelength. In another application, one or more of n-number of light sources 110 can have different wavelengths.

In another implementation, device 100 can have n-number of light sources 111 labeled as LED_{UVC1}-LED_{UVCn} that have a different wavelength from n-number of light sources 110. Each group of n-number of light sources 110 and n-number of light sources 111 can have their own switch (switch 118 and switch 119, respectively) to be turned manually on/off. In this implementation, the user turns on the one-way switch only for the n-number of light sources 110 or n-number of light sources 111 for the wavelengths of interest. This would allow device 100 to have multiple different light sources 110 with specifically tuned wavelengths of interest.

Each light source 110 can be controlled by a controller 114. Controller 114 ensures a constant current to light source 110. As light source 110 heats up from the current flow, the resistance of light source 110 tends to decrease. A decrease in the resistance with a constant voltage drop causes current to increase. To prevent this thermal runaway, controller 114 maintains a constant current to light source 110 throughout the temperature range. Controller 114 can be implemented as a single-output constant current LED driver driven by a milliamp current that is reducible by an analog voltage or a pulse-width modulated signal to Controller 114.

UVC light from light source 110 is not visible to the human eye. This can cause challenges to use and safety. To address these issues, n-number of light sources 110 and 111 are bounded on opposite sides by a pair of visible light sources 112. Visible light sources 112 can be implemented as extremely bright LEDs that shine a visible light on each side of n-number of light sources 110 so that the user is discouraged from looking directly at light source 110. The user can also see where the light from n-number of non-visible light sources 110 is being applied. As with light source 110, visible light sources 112 can be controlled by their own controller 114.

A duty cycle controller 120 can also be provided with device 100. Duty cycle controller 120 varies the on/off time and pulsating rate of light source 110 and light source 112. Duty cycle controller 120 can comprise an oscillator 122 controlled by a variable resistive element 124. Oscillator 122 can be implemented as a variable frequency square wave generator, such as the 555 variable frequency oscillator. Variable resistive element 124 can be a rheostat or potentiometer.

In one implementation, duty cycle controller 120 can vary the on/off time of light source 110 and light source 112 from 1 HZ to 7.5 Hz that corresponds to 0.1 seconds on and 0.9 seconds off to 0.1 seconds on to 0.033 seconds off. Of course any range between these ranges or any other range of duty cycling the on/off time of light source 110 and light source 112 is applicable. The variation can be dependent upon a ratio that ameliorates or intensifies cellular healing or growth in the surrounding tissue vs. inhibition of microbes.

A power source 116 is provided to drive each controller 114. In an embodiment, device 100 is powered by a dc power source, which can be in the form of multiple AA batteries. Power can be selectively applied to each controller 114 by a push-button switch 118 or push button switch 119. Switch 118 and switch 119 prevents device from being inadvertently left on. Those skilled in the art will recognize, however, that any type of switch or power source can be used.

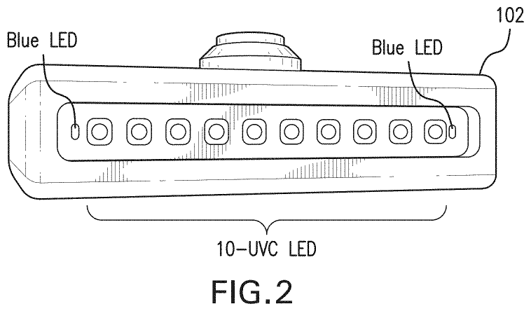

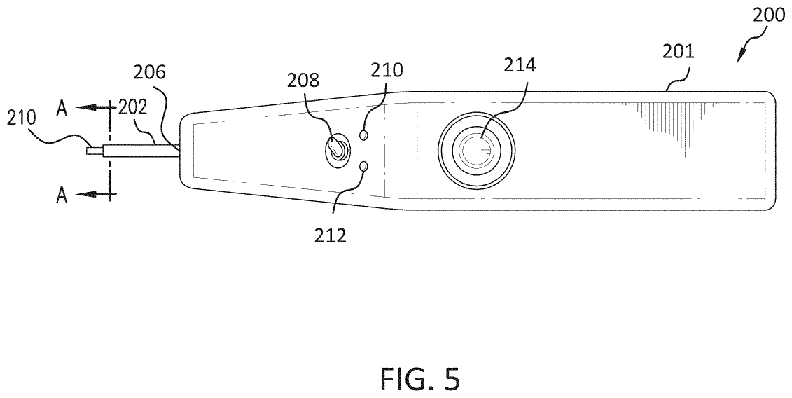

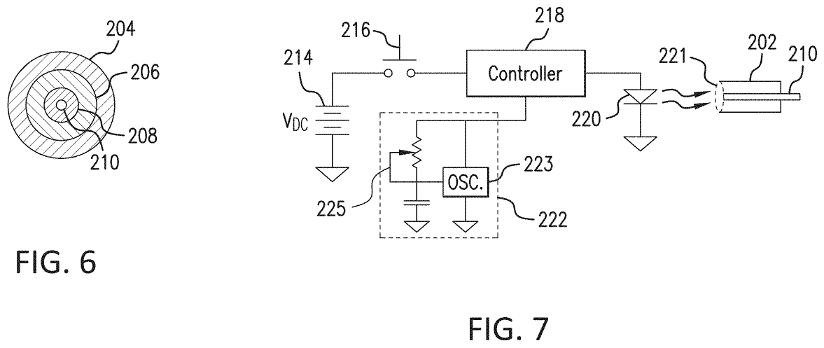

Device 100 can be implemented as a portable, handheld device, as shown in FIGS. 1-3. This means, device 100 can be implemented in a wide-variety of shapes. The general requirements for such shapes are that they are large enough to encompass the electronic circuitry, as discussed above, and that have an ergonomic and utilitarian shape so that it can be handled and used effectively. While the illustrated embodiment is implemented in a generally rectangular cuboid shape, device 100 can take have a more rounded form or shaped as a wand or a fiber optic strand. The fiber optic strand can be used “invasively” through insertion into open tissue such as a knee joint, fistula, tumor, etc., with the device held firmly or slowly pulled through the volume of tissue for disinfecting the tissue as herein described. This implementation is described more specifically in connection with FIG. 5.

Device 100 comprises of a housing 102 for storing the electronic circuitry and providing the ergonomically suitable feel for device 100. Housing 102 can comprise a removable cover 104 that slides on and off with respect to housing 102 for easy access to power source 116.

Housing 102 has a front-face 106 which is oriented toward the area of interest. Front-face 106 has a depression 108 that can extend along front-face 106 a sufficient length to provide space for each light source 110, 111 and visible light source 112. When device 100 is oriented toward the area of interest and switch 118, 119 is pressed, visible light from visible light source 112 on opposite sides of depression 108 illuminates the opposite sides of device 110 in proximity on the area of interest while UVC from light source 110, 111 impinges on the area of interest.

As previously mentioned, application of light from device 110 is a function of the wavelength of light source 110, the intensity, and the time duration of the application. The specific effect of the selected wavelength, power, and time duration is determined a priori by a clinically-generated encyclopedia of cellular effects versus light application. The effects can be wide-ranging, from destruction of cells to growth enhancement to structural stimulation, depending on the specifics of the light application.

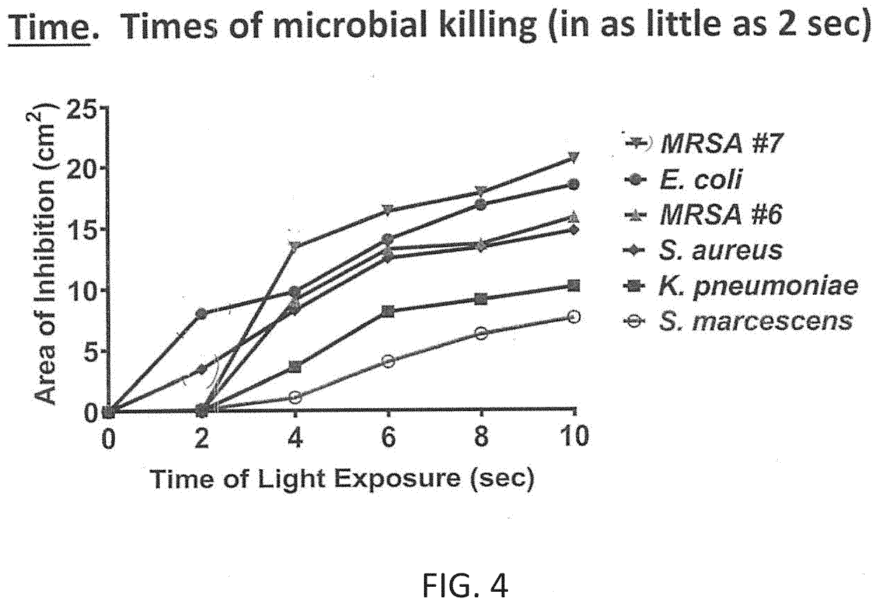

FIG. 4 shows a graph of area of inhibition versus time duration for six common microbes. This graph shows that device 100 kills e-coli and K. peneumoniae in as little as two seconds with total kill in ten seconds. Device 100 kills methicillin-resistant Staphylococcus aureus (MRSA) ##6, 7, S. aureus, and S. marcescens in as little as four seconds with substantial inhibition in ten seconds. These tests also determined that device 100 for light source 110 was most effective at a distance of 1-2 cm from the inhibition area. The testing was also done with device 100 with each light source 110 being a UVC LED with a 280 nm wavelength and having eight milliwatts of output power and half-power output pattern of 120° relative to the zenith of each LED.

FIG. 5 illustrates an alternative implementation to disinfect a volume of tissue. Shown is a device 200 with a housing 201, a light conduit 202, an attachment mechanism 206, a toggle switch 208, indicator elements 210, 212, and an electromagnetic energy delivery switch 214. Light conduit 202 implemented as, for example, hollow tube, fiberoptic strand, etc., can be inserted into a cellular volume and positioned so as to apply light in the selected volume to treat an area of interest. One or more such light conduits can be moved about the volume so as to apply light power proportionally throughout the volume to achieve a particular stimulation.

Light conduit 202 in the form of a plastic fiber optic strand is disclosed. Section A-A shows light conduit 202. Light conduit 202 can comprise an outer jacket 204, surrounding a buffer layer 206, surrounding a cladding layer 208, surrounding a core 210. The end of light conduit 202 has all the outer layers removed leaving only core 210 to apply therapeutic light outward in a 3D volume. Removing cladding layer 208 is counter-intuitive to optical fiber applications, since cladding layer traps the light in the core so that it exits as a point source of light. With cladding layer removed toward the distal end of light conduit 202, therapeutic light sprays outward in three dimensions.

Light conduit 202 must also be made plastic or a non-thermal conductive material. A glass or metal light conduit, for example, in the form of fiberoptics conducts heat. It has been discovered that heat has a detrimental effect cellular structure. So, while UVC light has the microbial killing benefits discussed above to disinfect a wound, heat will damage the surrounding cells to interfere with the healing effect.

FIG. 7 shows the electrical schematic for device 200, which is similar in operation to Device 100. Device 200 contains a power source 214 in series with switch 114 for providing power to a controller 218 which drives a light source 220 that is illustrated as a UVC LED. Controller 218 ensures a constant current to light source 220. As light source 220 heats up from the current flow, the resistance of light source 220 tends to decrease. A decrease in the resistance with a constant voltage drop causes current to increase. To prevent this thermal runaway, controller 218 maintains a constant current to light source 220 throughout the temperature range. Controller 218 can be implemented as a femtobucket, which is a constant current LED driver.

Controller 218 can also have a duty cycle control 222 that is manually adjustable by a rotating knob that adjusts the on/off time and pulsating rate in a manner. Duty cycle controller 222 varies the on/off time and pulsating rate of light source 220. Duty cycle controller 222 can comprise an oscillator 223 controlled by a variable resistive element 225. Oscillator 223 can be implemented as a variable frequency square wave generator, such as the 555 variable frequency oscillator. Variable resistive element 225 can be a rheostat or potentiometer.

In one implementation, duty cycle controller 120 can vary the on/off time of light source 220 from 1 HZ to 7.5 Hz that corresponds to 0.1 seconds on and 0.9 seconds off to 0.1 seconds on to 0.033 seconds off. Of course any range between these ranges or any other range of duty cycling the on/off time of light source 220 is applicable. The variation can be dependent upon a ratio that ameliorates or intensifies cellular healing or growth in the surrounding tissue vs. inhibition of microbes.

A power source 214 is provided to drive controller 218. In an embodiment, device 200 is powered by a dc power source, which can be in the form of multiple AA batteries. Power can be selectively applied to controller 218 by a switch 216. A switch 216 in the form of a push button prevents device from being inadvertently left on. Those skilled in the art will recognize, however, that any type of switch can be used.

A lens 221 is provided between light source 220 and the input of light conduit 202 for focusing the light from light source 220 into light conduit 202. Lens 221 reduces the amount of stray radiation into device 200 for more accurate determination of the amount of UVC radiation being applied to the treatment volume. It should also be understood that light source 220 may generate heat which has a negative effect on the circuitry as well as the therapeutic efficacy of device 100. Light conduit 221 serves to distance light source 220 from the application area, especially given that it is made of plastic or a non-thermal conductive material. Heat can also be moved away from light source 220 toward the handle of device 100 by one or more heatsinks.

It has also been shown that short exposure to UVC light from device 100 in the 2-10 second range with the intensity and wavelengths described herein has a therapeutic effect on tissue growth and cellular re-generation. This surprising result is counter-intuitive because it has been universally thought that UVC light damages cells and is carcinogenic with enough exposure. Device 100 has been shown to both kill the most common types of microbes and stimulate tissue regrowth.

Those skilled in the art will also recognize that UVC light sources described herein with respect to device 100 can be implemented as a light emitting diode, solid state laser, microwave generated UV plasma, or any type of fixed or tunable wavelength source that meets the design requirements. As device characteristics improve the spectral distribution can be narrowed. While wavelength between 200 to 280 nm (inclusive). The selected wavelength of the light source may have a narrow spectral output centered around a specific wavelength of, for example, ±10 nm. a wavelength of 265 nm is generally accepted as the optimum as it is the peak of the DNA absorption curve as averaged for most germs, more specific wavelengths can be used to target specific germs. In this regard, this means that, for example, device 100 can have light sources 110 at one frequency and light source 111 at another frequency. This also means the light sources 110 could have a mix of frequencies to specifically target a range of germs with each frequency selected for maximum absorption by the germs DNA.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

We claim:

1. A device for disinfecting and promoting tissue growth in an area of interest, the device comprising:
a housing;
a UVC emitting light source combined to the housing for emitting UVC light outside the housing onto the area of interest; and
a controller combined to the UVC light source for controlling the intensity of the UVC light emitted onto the area of interest.

2. The device of claim 1, wherein the housing further comprises a front-facing surface for orienting toward the area of interest, wherein the front-facing surface comprises of a depression that has positioned therein the UVC emitting light source.

3. The device of claim 2, and further comprising a pair of visible light sources positioned in the depression of the front-facing surface of the housing on opposite sides of the UVC emitting light source for providing visible lighted boundaries for the UVC light.

4. The device of claim 1, wherein the controller further comprises of a power source for providing power to the UVC emitting light source and a switch positioned electrically between the power source and the UVC emitting light source for selectively turning on and off the UVC emitting light source.

5. The device of claim 1, wherein the UVC emitting light source is a UVC light-emitting diode (LED).

6. The device of claim 5, further comprising a constant current device electrically connected between the power source and the UVC LED for controlling the current to the UVC LED.

7. The device of claim 6, and further comprising a plurality of parallel-connected UVC LEDs that are each controlled by one of a corresponding plurality of parallel-connected constant current devices.

8. The device of claim 7, and further comprising a pair of visible light sources on opposite sides of the plurality of UVC LEDs for providing visible boundaries for the UVC light and warning of the UVC light.

9. The device of claim 1, and further comprising a plurality of UVC emitting light sources with each having a spectral output centered around a different specific wavelength.

10. The device of claim 9, wherein each of the plurality of UVC emitting light sources is connected to its own controller for independent on/off control.

11. The device of claim 6, wherein the constant current device is a milliamp current to the constant current device, which is reducible by an analog voltage or a pulse-width modulated signal to the constant current device, and where the light source is a light emitting diode and the constant current device provides a constant current to the light emitting diode throughout the temperature range of the light emitting diode.

12. The device of claim 11, wherein the controller further comprises of a duty cycle controller to modify a pulsating on/off rate of the light source.

13. The device of claim 1, and further comprising: a plurality of UVC emitting light sources connected in parallel; a front-facing surface with a depression extending along the front-facing surface with the plurality of UVC emitting light sources positioned in a row in the depression; a pair of visible light sources positioned on opposite sides of the UVC emitting light source for providing visible lighted boundaries for the UVC light; and a plurality of constant current devices each one of which is connected to a corresponding one of the plurality of UVC emitting light sources to provide a constant current to the corresponding UVC emitting light source throughout the temperature range of the UVC emitting light source.

14. The device of claim 1, and further comprising a light conduit in electromagnetic communication with the light source for communicating light from the light source to the area of interest.

15. The device of claim 14, wherein the light conduit comprises a core extending the length of the conduit; and a cladding surrounding the core up to a distal end of the core, wherein a surface of the core is exposed at the distal end, and wherein light from a light source that emits light propagates through the core and is emitted from the core along the distal end of the core.

16. The device of claim 15, wherein the light conduit is a plastic fiberoptic conduit comprising a cladding that has been removed from the distal end so that light is emitted radially and axially relative to the distal end of the conduit for insertion into a volume of interest for treatment.

17. The device of claim 16, wherein the light conduit comprises a cylindrical distal end and wherein light radiates from the cylindrical distal end in three dimensions, and wherein the light conduit comprises of a transverse core that channels light from the light source therethrough and a cladding layer surrounding the core to the beginning of the cylindrical distal end to block light from exiting the core before reaching the cylindrical distal end, wherein the cladding layer is removed from the light conduit at the cylindrical distal end, and wherein the cylindrical distal end is a portion of the core of the light conduit without the cladding layer.

18. The device of claim 17, wherein the UVC emitting light source is a UVC light-emitting diode (LED).

19. The device of claim 18, further comprising a constant current device electrically connected between the power source and the UVC LED for controlling the current to the UVC LED.

20. The device of claim 19, wherein the constant current device is a milliamp current to the constant current device, which is reducible by an analog voltage or a pulse-width modulated signal to the constant current device, and where the light source is a light emitting diode and the constant current device provides a constant current to the light emitting diode throughout the temperature range of the light emitting diode, and wherein the controller further comprises of a duty cycle controller to modify a pulsating on/off rate of the light source.
